(19) United States
(12) Patent Application Publication
(10) Pub. No.: US 2026/0249286 A1
(43) Pub. Date: Aug. 27, 2026

(54) LATERAL FLOW DEVICE

(71) Applicant: SECRETARY OF STATE FOR HEALTH AND SOCIAL CARE, London (GB)

(72) Inventors: Jamie STONE, Hertfordshire (GB); Theo KING, Hertfordshire (GB); Holly PALMER, Hertfordshire (GB); John RIPPETH, Hertfordshire (GB)

(73) Assignee: SECRETARY OF STATE FOR HEALTH AND SOCIAL CARE, London (GB)

(21) Appl. No.: 18/993,862

(22) PCT Filed: Jul. 13, 2023
(86) PCT No.: PCT/GB2023/051832

§ 371 Date: Jan. 13, 2025

(30) Foreign Application Priority Data
Jul. 14, 2022 (GB) 2210335.2

(51) Int. Cl. B01L 3/00; G01N 33/543; G01N 33/569

(52) U.S. Cl. CPC B01L 3/5023; B01L 3/5029; G01N 33/54388; G01N 33/56983; B01L 2200/026; B01L 2200/0647; B01L 2200/0689; B01L 2200/087; B01L 2200/12; B01L 2300/025; B01L 2300/044; B01L 2300/069; B01L 2300/0825; B01L 2300/0832; B01L 2400/0406; G01N 2333/165; G01N 2469/10

(57) ABSTRACT

A lateral flow device comprising a body part and a removable swab, wherein the swab comprises a shaft having a proximal end and a distal end, with an absorbent head at the distal end, and the body part comprises: an inlet region for holding the swab prior to use, and for receiving the swab in use, during the execution of a lateral flow test; a fluid chamber containing a quantity of extraction solution; an outlet conduit; and a lateral flow assay strip; wherein the fluid chamber is arranged to open upon forcible insertion of the absorbent head into the body part, through the inlet region, in use, to cause the absorbent head to be treated with the extraction solution, and to cause the extraction solution to enter the outlet conduit; and wherein the outlet conduit includes, or is in fluid communication with, a proximal end of the lateral flow assay strip, such that, in use, extraction solution that enters the outlet conduit is able to reach the proximal end of the lateral flow assay strip, and thence flow along the lateral flow assay strip to execute a lateral flow test. Also provided is a method of performing a lateral flow test using such a lateral flow device.

### FIELD OF THE INVENTION

This invention relates to a lateral flow device, for use in performing a lateral flow test on a sample of liquid, to detect the presence (or absence) of a target substance therein. The liquid may for example be a sample of oral or nasal fluid (e.g. saliva or mucosal excretions), or blood, urine, or another bodily fluid. The target substance may for example be an antigen, such as a viral protein.

The invention is particularly suitable for determining the presence (or absence) of the SARS-COV-2 virus among members of the general population, for example as part of a nationwide “self-test” testing programme, by detecting the presence (or absence) of a Covid-19 antigen. However, the invention is by no means limited to this purpose, and may instead be used to detect the presence (or absence) of other pathogens, antigens, proteins, target molecules (e.g. hormones), or other target substances.

### BACKGROUND TO THE INVENTION

A lateral flow device may be used to test a sample of bodily fluid from a person (a subject), to provide a rapid indication as to the presence (or absence) of a target substance therein. The test result is typically obtained in a matter of minutes from the lateral flow device itself, without the need for additional equipment or laboratory-based processing. The sample may be obtained by a user of the tube in respect of themselves as the subject (as in the case of a “self-test”), or from another person (e.g. by an adult in respect of a child, or by a healthcare professional in respect of a patient, etc).

Particularly when testing oral or nasal fluids, e.g. for the presence of the SARS-COV-2 virus, a lateral flow device is typically provided as part of a “rapid antigen” lateral flow test kit, e.g. for determining the presence of a Covid-19 antigen. Such a test kit typically includes, as separate articles:
an extraction tube (that typically incorporates a cap having a dropper function),
a container (e.g. sachet or bottle) of extraction solution,
a swab (having an absorbent head on the end of a shaft), and
the lateral flow device itself (that typically comprises a lateral flow assay strip mounted within a plastic housing).

The swab is typically provided in its own sealed wrapper, and similarly the lateral flow device is typically provided in its own sealed packaging, to ensure that neither article is contaminated prior to use. The extraction test tube may also be provided in its own separate packaging.

In use, the lateral flow device is removed from its packaging and placed onto a clean flat surface, ready for use. The extraction tube is also removed from any packaging and placed on a clean surface or support (e.g. using a tube holder provided as part of the kit). The extraction solution (which may also be known as “buffer solution”) is transferred from its container into the extraction tube, with the user needing to take care not to spill any of the solution or to contaminate either the solution or the tube.

The swab is then removed from its wrapper, with the user needing to take care not to touch or otherwise contaminate the absorbent head. The swab is then used to collect a fluid sample from the subject using the absorbent head, e.g. from their nasal passages and/or the back of their throat.

The absorbent head is then introduced into the extraction solution in the extraction tube, with the user pressing or squeezing the absorbent head against the walls of the extraction tube, to transfer the collected fluid sample-including the target substance (e.g. Covid-19 antigen), if present-into the extraction solution. The resulting solution containing the fluid sample may be referred to herein as a test solution. Again, care needs to be taken not to contaminate the extraction solution or the resulting test solution during this process.

A specific quantity (e.g. a couple of drops) of the test solution is then transferred from the extraction tube to an inlet of the lateral flow device (e.g. by means of a dropper cap on the extraction tube, if such a cap is provided), again taking care not to contaminate either the test solution or the lateral flow device during this process, or to lose the test solution.

From the inlet of the lateral flow device, the test solution is then transferred by means of capillary flow along the assay strip, from a proximal end of the assay strip (coincident with said inlet) towards a distal end of the assay strip. This results in the generation of a positive test line in a first indicator region along the assay strip if the target substance (e.g. Covid-19 antigen) is present, and typically, further along the assay strip, the generation of a control line in a second indicator region, to confirm that the test solution has flowed along the assay strip correctly.

More generally, the assay strip has a proximal end (for receiving solution to be tested), a distal end (towards which the solution flows), and one or more indicator regions between the proximal end and the distal end, for determining the presence (or absence) of the target substance.

In practice, as those skilled in the art will appreciate, the composition of the assay strip will vary according to the target substance being detected in the test solution. Suitable assay strip compositions will be well known to those skilled in the art and need not be described in detail herein. However, in overview, such assay strips are designed to convey the test solution along the surface of a pad possessing reactive molecules that show a visual positive or negative result. The pads are based on a series of capillary beds, such as pieces of porous paper or polymer (e.g. a microstructured polymer or sintered polymer), and are able to transport the test solution spontaneously. Typically an assay strip includes, in sequence, a sample pad, a conjugate pad, and a wicking pad. The sample pad, at the proximal end of the assay strip and coincident with said inlet, acts to absorb the test solution and to hold an excess of the test solution. From there, the test solution flows to the conjugate pad which possesses conjugates in a salt-sugar matrix, wherein the conjugates typically comprise freeze-dried bio-active particles. The conjugate pad has the necessary reagents to produce a chemical reaction between the target substance (e.g. antigen) and a chemical partner of the target substance (e.g. antibody) that has been immobilized on the surface of the particles. This marks target particles as they pass through the pad and continue across to the test and control lines, in respective indicator regions. The test line provides a coloured indication if the target substance is present. The control line contains affinity ligands which indicate whether the test solution has flowed past, and the particles in the conjugate pad are active. After passing these reaction zones, the test solution enters the wicking pad, that essentially acts as a waste receptacle. will be appreciated that there are a number of potential shortcomings or disadvantages associated with the use of a conventional lateral flow device of the type as outlined above, including:
the kit being made up of a number of separate articles, which can be bulky to store and inconvenient to carry about one's person;
the need to unwrap the various separate articles which make up the kit, which can be awkward (e.g. for people having poor manual dexterity) and time consuming, and can result in the generation of a significant amount of packaging waste;
the need to prepare a clean flat surface on which to place the lateral flow device and extraction tube;
the need to transfer the extraction solution carefully from its container into the extraction tube, without spilling any of the solution or contaminating either the extraction solution or the tube;
the need to transfer the collected fluid sample from the swab to the extraction solution (e.g. by squeezing), without contaminating the extraction solution or the resulting test solution; and
the need to transfer a specific quantity (i.e. a specific number of drops) of the test solution to the lateral flow device, without contaminating the test solution or the lateral flow device, and without losing the test solution.

As a result of the above shortcomings, conventional lateral flow tests cannot readily be performed in locations away from a domestic (or similar) setting. In particular, conventional lateral flow tests cannot readily be performed by a user away from home, when standing up, e.g. in a queue at an airport, train station, theatre, cinema, concert hall, conference hall, retail establishment, place of religious worship, sports venue, bar, nightclub, dancehall or other venue at which many other people will potentially be present, where the user, if infected with a virus (e.g. SARS-COV-2), would risk transmitting the virus to others (potentially many others) within the venue. It will be appreciated that, at such venues, it would be desirable to be able to establish, through on-the-spot lateral flow testing, whether a person is infected with such a virus and, if so, not to admit them to the venue. Conversely, establishing that people at the venue are not infected with such a virus enables them to mix more freely and comfortably within the venue, with low risk of spreading or catching the virus.

Moreover, from the above, it will be appreciated that conventional lateral flow tests are susceptible to contamination or other issues (e.g. liquid loss) when carrying out the tests, which can in turn affect the accuracy of the results. Furthermore, the need to unwrap the various articles that make up the test kit can be awkward and can lead to the generation of an appreciable amount of waste.

There is therefore a desire for a lateral flow device that people can use to self-test for the presence (or absence) of a target substance, such as the SARS-COV-2 virus, that overcomes one or more of the above disadvantages and enables testing to be performed more readily by users, including away from home.

### SUMMARY OF THE INVENTION

According to a first aspect of the present invention there is provided a lateral flow device as defined in Claim 1 of the appended claims. A method of performing a lateral flow test using such a lateral flow device is also provided. Optional features, and details of certain embodiments, are set out in the appended dependent claims. Thus, according to a first aspect of the present invention there is provided a lateral flow device comprising a body part and a removable swab, wherein the swab comprises a shaft having a proximal end and a distal end, with an absorbent head at the distal end, and the body part comprises:
an inlet region for holding the swab prior to use, and for receiving the swab in use, during the execution of a lateral flow test;
a fluid chamber containing a quantity of extraction solution (also referred to as “buffer solution” herein);
an outlet conduit; and
a lateral flow assay strip;
wherein the fluid chamber is arranged to open upon forcible insertion of the absorbent head into the body part, through the inlet region, in use, to cause the absorbent head to be treated with the extraction solution (thereby mixing the extraction solution with a fluid sample collected on the absorbent head), and to cause the extraction solution (with the fluid sample mixed therein) to enter the outlet conduit; and
wherein the outlet conduit includes, or is in fluid communication with, a proximal end of the lateral flow assay strip, such that, in use, extraction solution (including the fluid sample) that enters the outlet conduit is able to reach the proximal end of the lateral flow assay strip, and thence flow along the lateral flow assay strip to execute a lateral flow test.

Accordingly, the body part and the swab may conveniently be provided as a single portable article, prior to use. Moreover, by including the fluid chamber and the lateral flow assay strip, the body part integrally incorporates all the components necessary to perform a lateral flow test in respect of a fluid sample collected by the absorbent head of the swab. As a consequence, the device is quick and easy to use, and there is no need for a clean flat surface on which to place the device. In turn, this enables the device to be used away from the user's home, including when the user is standing up-e.g. in a queue at an airport, train station, theatre, cinema, concert hall, conference hall, retail establishment, place of religious worship, sports venue, bar, nightclub, dancehall or other such venue. Moreover, as a result of the way in which fluids are automatically conveyed within the device, there is no need for the user to carry out intricate processing steps themselves, and the risk of contamination (of the fluid sample, extraction solution, swab, or other components) is significantly reduced.

The fluid chamber may have a first rupturable seal at a proximal end of the fluid chamber, and a second rupturable seal at a distal end of the fluid chamber; wherein, in use, the first seal of the fluid chamber is arranged to be penetrated by the absorbent head of the swab upon partial insertion of the swab into the body part, and to rupture; the second seal of the fluid chamber is arranged to be penetrated by the absorbent head of the swab upon further insertion of the swab into the body part, and to rupture; and the second seal of the fluid chamber is adjacent the outlet conduit, such that, upon rupturing the second seal in use, extraction solution is able to flow from the fluid chamber into the outlet conduit. Accordingly, such a fluid chamber may be readily opened in use, simply by the user inserting the swab (preferably simply driving the swab fully home within the body part) and thereby causing the first and second seals to rupture.

Preferably the inlet region, fluid chamber and outlet conduit are arranged colinearly within the body part, to permit the absorbent head of the swab to be inserted in a straight linear manner along the inlet region, through the fluid chamber, and into the outlet conduit.

Advantageously, the inlet region and outlet conduit may be integrally formed within the body part, and the fluid chamber may be formed as an initially-separate pre-filled component (essentially a cartridge) that is inserted into the body part during manufacture. This facilitates the manufacture of the lateral flow device, in particular with respect to the introduction of the extraction solution into the device. This also enables the sterility of the extraction solution to be assured, as it enables the fluid chamber to be pre-filled and sealed under carefully-controlled sterile conditions.

Beneficially, to define the extent to which the swab enters the body part when the swab is fully inserted (i.e. driven home) within the body part, the shaft may comprise a radially protruding part (e.g. flange) and the inlet region or the fluid chamber may comprise a stop (or other detent) against which the radially protruding part of the swab abuts (or engages) when the swab is fully inserted into the body part in use.

Advantageously, the length of the swab from the protruding part to the tip of the absorbent head may correspond to the distance along the body part from the stop to a point partway along the outlet conduit. As a consequence, the tip of the absorbent head will pass through the fluid chamber and emerge into the outlet conduit when the swab is fully inserted into the body part.

Preferably, the inlet region and the fluid chamber are cylindrical, to enable the swab (having a circular cross section) to be inserted in any orientation about its axis.

Optionally the internal diameter of the fluid chamber may be less than the diameter of the absorbent head, to cause the absorbent head to be radially compressed upon entering the fluid chamber and thereby aid the release of a fluid sample collected by the absorbent head in use.

Preferably the proximal end of the shaft comprises a handle head by means of which a user can push the swab into the body part in use. The handle head may have a wider diameter than the inlet region, the handle head having an underside which is shaped to abut the opening of the inlet region when the swab is fully inserted into the body part in use. Further, the underside of the handle head may be shaped to plug the opening of the inlet region when the swab is fully inserted into the body part in use.

Preferably the shaft and/or the inlet region comprise sealing means for forming a seal between the shaft and the inlet region upon insertion of the absorbent head into the body part (in particular, into the fluid chamber or beyond) in use. For example, the sealing means may comprise one or more fins formed on the shaft.

In the presently-preferred embodiment, the body part further comprises a housing in which at least a substantial part of the lateral flow assay strip is contained. The housing may have one or more openings through which one or more indicator regions of the lateral flow assay strip can be viewed.

Preferably the arrangement of the housing relative to the rest of the body part is broadly in the manner of a pen clip relative to the rest of a pen. That is to say, the housing (and the assay strip therein) doubles-back on the rest of the body part, making the overall device more compact, and also enabling the overall device to be clipped in a pocket prior to, or after, use. Indeed, the dimensions of the device may also be broadly similar to a typical pen.

Preferably the device further comprises a removable sheath that surrounds at least the absorbent head of the swab prior to use, thereby keeping the absorbent head clean and sterile prior to use. The sheath may comprise a barrier film wrapper, although in alternative embodiments the sheath may be substantially rigid. The shaft of the swab may comprise one or more ridges or fins (preferably a plurality thereof) around which a proximal end of the sheath may be engaged, prior to use. The proximal end of the sheath may be formed as a collar around the one or more protrusions, ridges or fins.

Advantageously, the proximal end of the sheath may be provided with a tab and/or perforations by means of which the sheath may be ripped, to enable it to be easily detached from the swab prior to use. The one or more protrusions, ridges or fins, around which the collar is formed, may also serve as sealing means for forming a seal between the shaft and the inlet region upon insertion of the absorbent head into the body part in use.

Preferably the diameter of the collar is greater than the diameter of the inlet region, thereby holding the sheath relative to the inlet region prior to use, and the length of the sheath from an underside of the collar to the distal end of the sheath is less than the length of the body part from the opening of the inlet region to the fluid chamber, thereby holding the swab clear of the fluid chamber prior to use.

Preferably the swab and/or the inlet region comprise gripping means for releasably gripping the swab within the inlet region prior to use. For instance, the gripping means may comprise one or more ridges or fins (preferably a plurality thereof) formed on the shaft.

According to a second aspect of the present invention there is provided a method of performing a lateral flow test using a lateral flow device according to the first aspect of the invention, the method comprising:
removing the swab from the inlet region of the body part;
collecting a fluid sample using the absorbent head; and
forcibly inserting the absorbent head into the body part, thereby causing the fluid chamber to open, the extraction solution to treat the absorbent head, and extraction solution containing the fluid sample to enter the outlet conduit, reach the proximal end of the lateral flow assay strip, and thence flow along the lateral flow assay strip to execute a lateral flow test.

When using a fluid chamber as described above, having a first rupturable seal at a proximal end of the fluid chamber, and a second rupturable seal at a distal end of the fluid chamber, the first seal of the fluid chamber is penetrated by the absorbent head of the swab upon partial insertion of the swab into the body part, and ruptures; and the second seal of the fluid chamber is penetrated by the absorbent head of the swab upon further insertion of the swab into the body part, and ruptures.

In the case of the swab being initially sheathed, the method further comprises removing the sheath from the swab, after the swab is removed from the inlet region of the body part, and before a fluid sample is collected using the absorbent head.

### BRIEF DESCRIPTION OF THE DRAWINGS

Embodiments of the invention will now be described, by way of example only, and with reference to the drawings in which:

FIG. 1 is a partial cutaway view of a lateral flow device prior to use, the lateral flow device including a removeable swab and a body part having an integral lateral flow assay strip, the swab being sheathed and releasably held within an inlet region of the body part prior to use;

FIGS. 2 and 3 are perspective views of the lateral flow device of FIG. 1 prior to use;

FIGS. 4, 5 and 6 are side, top and bottom views, respectively, of the lateral flow device of FIG. 1 prior to use;

FIGS. 7 and 8 are end views of the lateral flow device of FIG. 1 prior to use;

FIG. 9 is a partial cutaway view of the lateral flow device of FIG. 1 in use, with the swab having been unsheathed and, following collection of a fluid sample, partially inserted into the body part;

FIG. 10 is a partial cutaway view of the lateral flow device of FIG. 1 in use, a moment later than is depicted in FIG. 9, with the swab having been fully inserted into the body part;

FIG. 11 is a cross-sectional view of the lateral flow device of (and corresponding to) FIG. 1, prior to use;

FIG. 12 is a cross-sectional view of the lateral flow device of (and corresponding to) FIG. 10, in use; and

FIGS. 13 and 14 are perspective and side views, respectively, of the lateral flow device of FIGS. 10 and 12, in use.

In the figures, like elements are indicated by like reference numerals throughout.

### DETAILED DESCRIPTION OF PREFERRED EMBODIMENTS

The present embodiments represent the best ways known to the Applicant of putting the invention into practice. However, they are not the only ways in which this can be achieved.

### Initial Overview

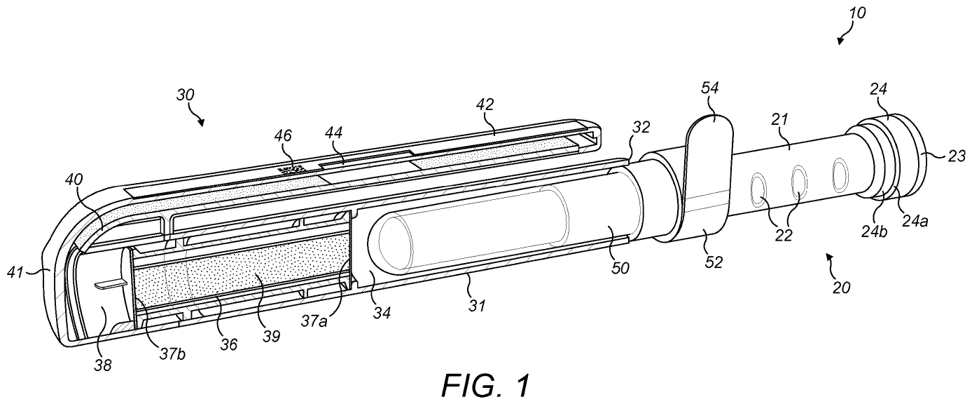

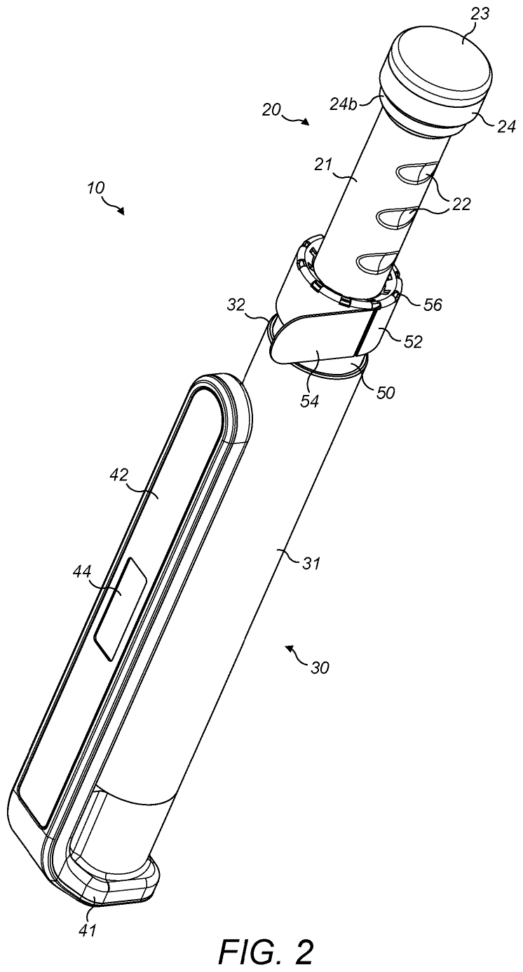

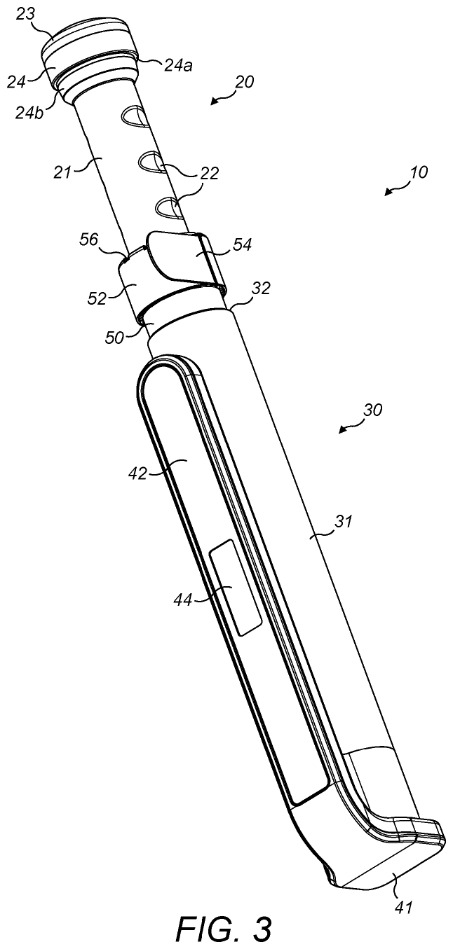

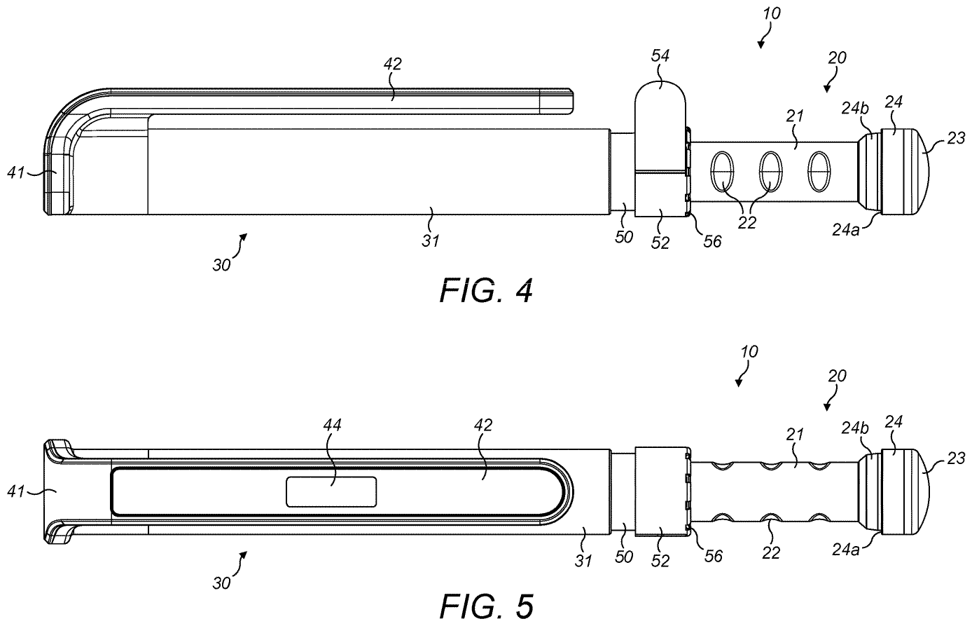

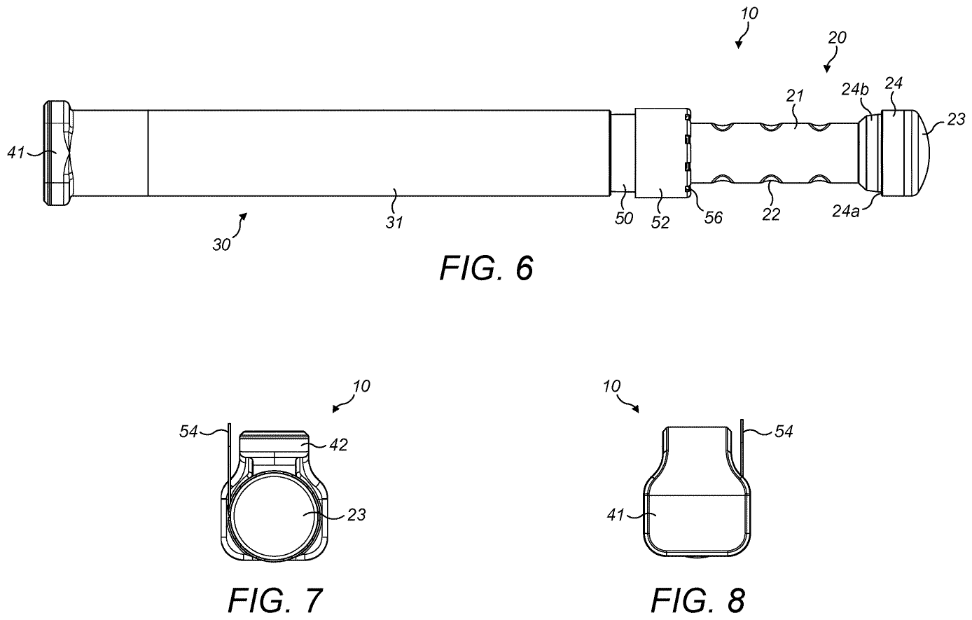

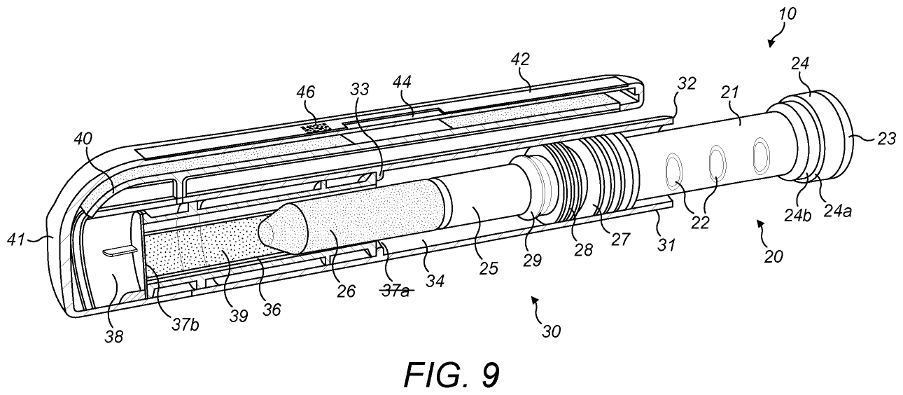

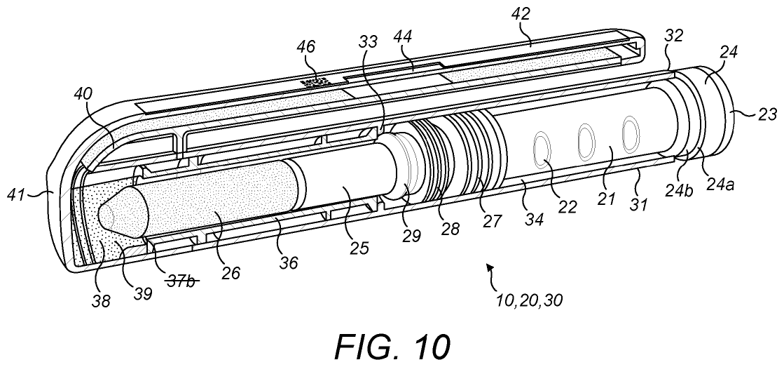

With reference initially to FIG. 1, the present disclosure provides a lateral flow device 10 comprising a body part 30 and a removable swab 20. Turning briefly to FIGS. 9 and 10 (since the swab 20 is partly covered by a sheath 50 in FIG. 1, obscuring some of the features of the swab 20), the swab 20 comprises a shaft 21, 25 having a proximal end and a distal end. An absorbent head 26 is provided at the distal end of the swab 20, suitable for collecting a fluid sample in use (e.g. nasal and/or oral fluid, from the user's nasal passages and/or the back of their throat, with the fluid sample potentially containing a target substance such as a particular antigen, if present).

In the illustrated embodiment of FIG. 1, the lateral flow device 10 is preferably supplied to the user with the swab 20 partially inserted into an inlet region 34 of the body part 30, with the swab 20 partly covered by the removable sheath 50.

Alternatively, the swab 20 may in principle be supplied separately from the body part 30, although supplying the swab 20 partially inserted into the body part 30 provides the advantage that the user is conveniently provided with a single overall device 10, comprising the body part 30 and the swab 20, ready for use as and when required.

As illustrated in FIG. 1, the lateral flow device 10 is preferably supplied to the user with at least the absorbent head 26 of the swab 20 covered by the removable sheath 50, to keep the absorbent head 26 sterile and clean prior to use. The sheath 50 is removed by the user just before they use the swab 20 to collect a fluid sample using the absorbent head 26.

Meanwhile, the body part 30 integrally incorporates all the components necessary to perform a lateral flow test in respect of a fluid sample collected by the absorbent head 26 of the swab 20, including a quantity of extraction solution 39, and a lateral flow assay strip 40.

In more detail, the body part 30 includes, in sequence, four main parts, namely:
an inlet region 34 (having an opening 32) for holding the swab 20 prior to use, and for receiving the swab 20 in use, for executing a lateral flow test;
a fluid chamber 36 pre-filled at the time of manufacture with a quantity of extraction solution (also known as “buffer solution”) 39;
an outlet conduit 38; and
a lateral flow assay strip 40.

The overall device 10 preferably has a slim and compact overall configuration, broadly similar to that of a pen, enabling the user to conveniently carry the device 10 about their person (e.g. in a jacket or coat pocket, or handbag, etc.). The overall device 10 is therefore well-suited to on-the-spot testing by a user away from home, e.g. in a queue at an airport, train station, theatre, cinema, concert hall, conference hall, retail establishment, place of religious worship, sports venue, bar, nightclub, dancehall or other venue at which many other people will potentially be present, where the user, if infected with a virus (e.g. SARS-COV-2), would risk transmitting the virus to others (potentially many others) within the venue.

In a general sense, and as illustrated by way of example in FIGS. 9, 10 and 12, the fluid chamber 36 is arranged to open upon forcible insertion of the absorbent head 26 of the swab 20 into the body part 30, through the inlet region 34, during a test, to cause the absorbent head 26 to be treated with the extraction solution 39. This has the effect of transferring the fluid sample collected by the absorbent head 26 (including a target substance, e.g. antigen, if present) into the extraction solution 39.

As shown in FIGS. 10 and 12, the extraction solution 39 (including the target substance, if present) also enters the outlet conduit 38. The outlet conduit 38 includes, or is in fluid communication with, a proximal end of the lateral flow assay strip 40, such that, in use, extraction solution 39 that enters the outlet conduit 38 is able to reach the proximal end of the assay strip 40, and thence flow along the assay strip 40 to execute a lateral flow test.

In the illustrated embodiment the fluid chamber 36 is initially manufactured as a separate component, essentially in the form of a cartridge that is pre-filled with the extraction solution 39. The pre-filled fluid chamber 36 is then inserted into the body part 30 during manufacture. The fluid chamber 36, which is rectangular in cross-section, has tubular side walls, a first rupturable seal 37a at one end (the proximal end when installed in the body part), and a second rupturable seal 37b at the other end (the distal end when installed in the body part). The side walls of the fluid chamber 36 may be made of any suitable plastics material, such as polypropylene, for instance. The first and second seals 37a, 37b may for example be made of metallised polymer film (e.g. metallised polypropylene or metallised polyethylene terephthalate). By way of example, aluminium may be used to metallise these films. The extraction solution 39 is contained between the first and second seals 37a, 37b. Preferably the fluid chamber 36 is substantially full of extraction solution 39. The volume of the extraction solution may be approximately 1 ml (1 cm³).

As shown in FIGS. 9, 10 and 12, with a fluid chamber 36 of this cartridge-like form, in use the above-described opening of the fluid chamber is effected by rupturing the first and second seals 37a, 37b by the absorbent head 26, by swift forcible insertion of the swab 20 deep into the body part 30, to the fullest extent permitted by the architecture of the device-in other words, by pushing the swab 20 fully home within the body part 30. The forcible insertion of the swab 20 is preferably performed as a single uninterrupted one-way insertion action, typically within a second or two, thus driving or ramming the swab 20 home within the body part 30.

More particularly, when the swab 20 is progressively inserted into the body part 30, the absorbent head 26 is first forced to enter the fluid chamber 36, penetrating the first seal 37a (and thus the numeral 37a is shown using strikethrough in FIG. 9); and then the tip of the absorbent head 26 is forced to emerge from the opposite end of the fluid chamber 36, penetrating the second seal 37b (and thus the numeral 37b is shown using strikethrough in FIG. 10).

In other words, the first seal 37a of the fluid chamber 36 is ruptured or burst by the absorbent head 26 upon partial insertion of the swab 20 into the body part 30, and then the second seal 37b of the fluid chamber 36 is ruptured or burst by the absorbent head 26 upon further insertion of the swab 20 into the body part 30 (i.e. as the swab 20 is fully pushed home).

In the present embodiment the rupturing of the first and second seals 37a, 37b of the fluid chamber 36 by the progressive advancement of the swab 20 is due to the absorbent head 26 contacting and applying pressure on each of the seals 37a, 37b in turn. However, in an alternative embodiment, rupture of the second seal 37b may occur as a result of pressure build-up in the extraction solution 39, before the absorbent head 26 actually reaches the second seal 37b.

The second seal 37b of the fluid chamber 36 is adjacent the outlet conduit 38, such that, upon rupturing the second seal 37b in use, extraction solution 39 (containing the collected fluid sample) is able to flow from the fluid chamber 36 into the outlet conduit 38, and thence reach the integral lateral flow assay strip 40.

As illustrated, preferably the inlet region 34, fluid chamber 36 and outlet conduit 38 are arranged colinearly within the body part 30, to permit the absorbent head 26 of the swab 20 to be inserted in a straight linear manner along the inlet region 34, through the fluid chamber 36, and into the outlet conduit 38.

It should be noted, however, that the above-described use of a cartridge-like fluid chamber 36, with rupturable first and second seals 37a, 37b at each end, is only one way by means of which the fluid chamber may be arranged to open upon insertion of the swab 20 into the body part 30. Alternative mechanisms are also possible, by which insertion of the swab 20 into the body part 30 will cause the fluid chamber to open, thereby treating the absorbent head 26 with extraction solution from within the fluid chamber. For example, a mechanical arrangement could be provided whereby insertion of the swab 20 into the body part 30 causes a flap on the fluid chamber to open, thereby releasing the extraction solution therein. Such a flap could be directly pushed open by the inserted swab 20, or could, for instance, be opened by means of a lever arrangement actuated by the swab 20 on insertion into the body part 30. In another example, the fluid chamber could take the form of a rupturable sac, filled with extraction solution, that is burst upon insertion of the swab 20, for instance.

It should also be noted that, whilst the illustrated embodiment uses a fluid chamber 36 that is arranged to open at both ends (by rupturing the first seal 37a and then the second seal 37b as the swab 20 is progressively inserted into the body part 30 and the absorbent head 26 passes through the fluid chamber 36), in alternative arrangements a single opening may be provided through which extraction solution within the fluid chamber is released upon insertion of the swab 20 into the body part 30, to cause the extraction solution to treat the absorbent head 26 of the swab 20, and to enable the extraction solution (containing the collected fluid sample) to enter the outlet conduit 38 and thereby reach the integral lateral flow assay strip 40.

### Further Details in Respect of the Swab 20

As mentioned above, and as shown for example in FIGS. 9 and 10, the swab 20 comprises a shaft 21, 25 having a proximal end and a distal end. The shaft 21, 25 is preferably cylindrical/rod-like in form, i.e. is circular in cross section, to enable it to be used (and inserted into the body part 30) in any orientation about its axis. An absorbent head 26 is provided at the distal end of the swab 20 (i.e. attached to region 25 of the shaft), suitable for collecting a fluid sample in use (e.g. nasal and/or oral fluid, from the user's nasal passages and/or the back of their throat). Further, in the illustrated embodiment, a handle head 24 is provided at the proximal end of the swab 20 (i.e. attached to region 21 of the shaft), by means of which the user can forcibly push the swab 20 into the body part 30 to execute the lateral flow test.

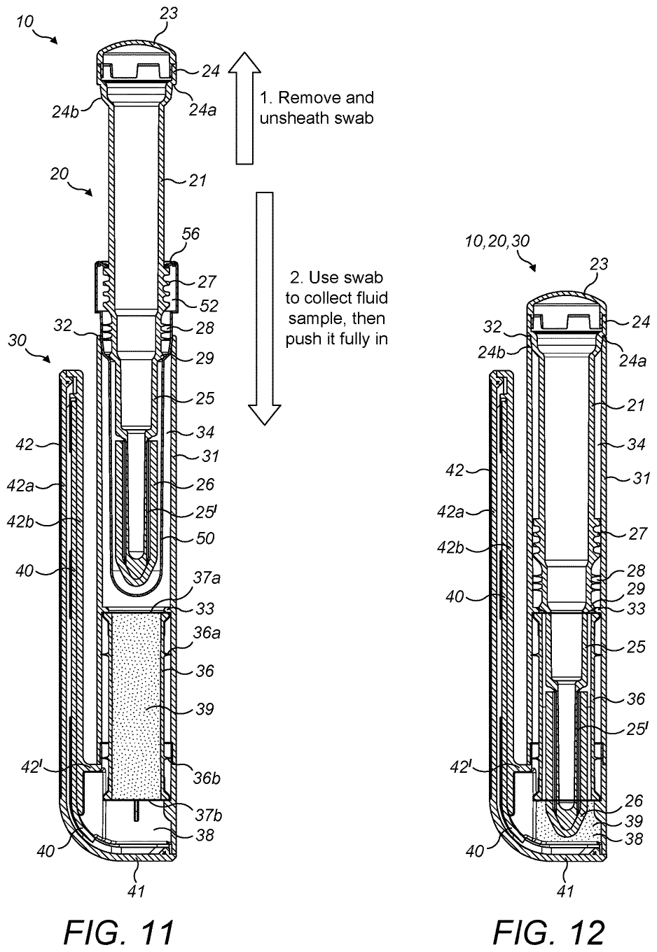

The absorbent head 26 may be made of any suitable material, such as a foam or cotton. As shown in FIGS. 11 and 12, in the illustrated embodiment the shaft 21, 25 is hollow, and the absorbent head 26 is mounted around a core 25′ that is a smaller-diameter continuation of region 25 of the shaft. Such a core 25′ reinforces the absorbent head 26, providing it with a degree of rigidity, which in turn facilitates the penetration of the first and second seals 37a, 37b during the execution of a test. However, in alternative embodiments, the absorbent head 26 could be formed without a reinforcing core 25′.

The shaft 21, 25 (and core 25′) is preferably made of a plastics material (e.g. by injection moulding) and may incorporate depressions 22 (in region 21 of the shaft) which act as finger grips, to facilitate manipulation of the swab 20 when collecting a fluid sample. The handle head 24 may be integrally formed with the rest of the shaft 21, 25.

As shown in FIGS. 1 and 11, during manufacture the swab 20 is fitted with a removable sheath 50 that surrounds at least the absorbent head 26 of the swab 20, to keep the absorbent head 26 clean and sterile prior to use. In the illustrated embodiment, the sheath 50 comprises a barrier film wrapper. To secure the film wrapper sheath 50, the shaft 21 of the swab 20 comprises one or more ridges or annular fins 27, or other protrusions, around which a proximal end of the sheath 50 is engaged, prior to use. In the illustrated embodiment, a plurality (first set) of such fins 27 is provided. In effect, the proximal end of the film wrapper sheath 50 is formed as a collar 52 around the fins 27 (or ridges or other protrusions).

The diameter of the collar 52 of the sheath 50 is greater than the diameter of the opening 32 of the inlet region 34, thereby holding the sheath 50 relative to the inlet region 34 prior to use (by virtue of the collar 52 remaining outside the opening 32 of the inlet region 34). Moreover, the length of the sheath 50 from an underside of the collar 52 to the distal end of the sheath 50 is less than the length of the body part 30 from the opening 32 of the inlet region 34 to the first seal 37a of the fluid chamber 36. Consequently, when the sheath 50 is in place, the swab 20 is held clear of the first seal 37a and does not risk rupturing it.

With reference to FIGS. 9, 10, 11 and 12, the one or more ridges or fins 27 (or other protrusions) around which the collar 52 is formed may also serve as sealing means for forming a seal between the shaft 21, 25 and the inlet region 34, upon insertion of the absorbent head 26 into the fluid chamber 36 during a test. In use, the sealing produced by the ridges or fins 27 (or the like) ensures that no extraction solution 39 leaks out once the first seal 37a is broken. Moreover, the ridges or fins 27 (or the like) may also advantageously act as a pressure release valve as the first seal 37a is broken and the swab 20 is pushed further into the body part 30.

With particular reference to FIG. 11, the swab 20 and/or the inlet region 34 may also comprise gripping means for releasably gripping (i.e. temporarily holding) the sheathed swab 20 within the inlet region 34 prior to use, e.g. at the opening 32 of the inlet region 34. By virtue of such gripping means, the swab 20 and the body part 30 are held together as a single article 10 until the user is ready to remove the swab 20 to begin performing a lateral flow test. As illustrated, the gripping means may comprise one or more ridges or annular fins 28 formed on the shaft 21, 25. Preferably the one or more ridges or fins 28 are moulded as an integral part of the shaft 21, 25. In the illustrated embodiment, a plurality of ridges or fins 28 are provided, and may be considered a second set (in addition to the first set 27 described above). As shown in FIG. 11, these ridges or fins 28 provide a light friction fit between themselves and the opening 32 of the inlet region 34, e.g. as a result of a small amount of elastic deformation of the ridges or fins 28. As shown in FIG. 11, this gripping effect occurs despite the presence of the film sheath 50 around the ridges or fins 28. Indeed, the presence of the sheath 50 may contribute to the gripping effect. Conversely, when the sheath 50 is subsequently removed in use, the gripping effect caused by only the ridges or fins 28 may be reduced, enabling the swab 20 to be inserted more easily into the body part 30 when executing the lateral flow test. In use, the ridges or fins 28 may also serve as additional sealing means for forming a seal between the shaft 21, 25 and the inlet region 34, upon insertion of the absorbent head 26 into the fluid chamber 36 during a test.

To prepare the device 10 for use, the user first removes the sheathed swab 20 from the body part 30 (overcoming the above-described gripping effect provided by the ridges or fins 28), then removes the sheath 50 from the swab 20 to expose the absorbent head 26.

With the illustrated embodiment, the removal of the film wrapper sheath 50 from the swab 20 is facilitated by the proximal end (collar 52) of the sheath 50 being provided with a tab 54 and a series of perforations 56, as shown for example in FIG. 1. By pulling on the tab 54 and causing the film between the perforations 56 to rupture, the sheath 50 can easily be ripped off the swab 20 and subsequently disposed of.

Once unsheathed, the swab 20 can then be used to collect a fluid sample on the absorbent head 26, with the user holding the swab 20 by the shaft 21 (aided by the depressions/finger grips 22) and naturally taking care not to contaminate the absorbent head 26 during the collection process.

As shown for example in FIG. 9, in the illustrated embodiment the swab 20 also incorporates a flange 29 (or other radially protruding part) on the shaft 25. Preferably the flange 29 is moulded as an integral part of the shaft 25. The position of the flange 29 defines the extent to which the swab 20 enters the body part 30 when the swab 20 is fully inserted (i.e. driven home), by virtue of the flange 29 abutting a stop 33 provided within the body part 30, as shown in FIGS. 10 and 12. A detent mechanism may also be provided, potentially emitting an audible click when the swab 20 is driven fully home within the body part 30.

From FIGS. 10 and 12, it will also be appreciated that the length of the swab 20 from the flange 29 (or other radially protruding part) to the tip of the absorbent head 26 corresponds to the distance along the body part 30 from the stop 33 to a point partway along the outlet conduit 38, i.e. beyond the second seal 37b.

Accordingly, the second seal 37b (as well as the first seal 37a) is broken upon full insertion of the swab 20 into the body part 30. Upon breaking the second seal 37b, any pressure build-up in the fluid chamber 36 (due to the progressive advancement of the swab 20) is released, and the extraction solution 39 (containing the collected fluid sample) is forced into the outlet conduit 38, from which it will then reach the proximal end of the lateral flow assay strip 40, and then make its way along the assay strip 40.

In view of the above, the present device may be considered to employ a “positive pressure displacement” principle as the absorbent head 26 of the swab 20 is advanced through the double-ended cartridge-like fluid chamber 36.

As shown for example in FIG. 1, the handle head 24 may be provided with a domed end 23 and may be somewhat enlarged relative to the rest of the swab 20. This facilitates the action of the user pushing the swab 20 forcibly (and yet comfortably) into the body part 10 in use.

It should also be noted that, in the illustrated embodiment, the handle head 24 has a wider diameter than the opening 32 of the inlet region 34. Moreover, as shown most clearly in FIGS. 10 and 12, the handle head 24 has an underside 24a that is shaped to abut the opening 32 of the inlet region 34 when the swab 20 is fully inserted into the body part 30 in use, and a portion 24b that is shaped to plug the opening 32 of the inlet region 34. To this end, in the illustrated embodiment the underside 24a of the handle head 24 is essentially step-like in shape, to enable it to squarely abut the opening end of the inlet region 34. The portion 24b is tapered, to guide the handle head 24 into centralised (coaxial) alignment with the opening 32 of the inlet region 34 and to plug the opening 32.

Accordingly, in the illustrated embodiment, when the swab 20 is fully inserted into the body part 30 (i.e. pushed home), two instances of abutment simultaneously occur-the abutment of the flange 29 against the stop 33, and the abutment of the underside 24a of the handle head 24 against the opening 32 of the inlet region 34. As shown in FIGS. 10 and 12, this results in the overall device 10 adopting a well-defined configuration in a well-controlled manner when the swab 20 is fully inserted into the body part 30, with the extent of longitudinal travel of the swab 20 relative to the body part 30 being precisely limited, and the swab 20 being concentrically (coaxially) aligned within the body part 30.

Moreover, from FIGS. 10 and 12, it can be seen that, at the point of full insertion of the swab 20 into the body part 30, the absorbent head 26 has passed through the first and second seals 37a, 37b of the fluid chamber 36, with some of the absorbent head 26 remaining within the fluid chamber 36 and the distal tip of the absorbent head 26 entering the outlet region 38. In this configuration, extraction solution 39 from the fluid chamber 36 is intermixed with the fluid sample collected by the absorbent head 26, enters the outlet region 38, and hence reaches the proximal end of the lateral flow assay strip 40, which is in (or is in fluid communication with) the outlet region 38. The lateral flow test is then executed by capillary flow of the extraction solution 39 (containing the collected fluid sample) along the assay strip 40.

### Further Details in Respect of the Body Part 30

As outlined above, the body part 30 includes, in sequence, four main parts, namely:
an inlet region 34 (having an opening 32) for holding the swab 20 prior to use, and for receiving the swab 20 in use, during the execution of a lateral flow test;
a fluid chamber 36 pre-filled at the time of manufacture with a quantity of extraction solution (also known as “buffer solution”) 39;
an outlet conduit 38; and
a lateral flow assay strip 40.

In the illustrated embodiment, the inlet region 34 and the fluid chamber 36 are both cylindrical in shape (i.e. have a circular internal cross section), and coaxially aligned with one another, to permit the swab 20 (which has a circular cross section) to be inserted in any orientation about its axis. The outlet conduit 38 may also be broadly cylindrical in shape. In use, as the first and second seals 37a, 37b of the fluid chamber 36 are ruptured, the inlet region 34, fluid chamber 36 and outlet conduit 38 coaxially form a continuous tubular channel through which the swab 20 passes. It will be appreciated that said continuous tubular channel might not have a constant internal diameter throughout. In particular, as illustrated, the fluid chamber 36 may have a smaller internal diameter than the inlet region 34 and outlet conduit 38 primarily because of the fluid chamber 36 being formed as an initially-separate pre-filled component.

In alternative embodiments, it is possible that at least part of the swab 20, and the internal cross section of the inlet region 34 and/or the fluid chamber 36, may have a non-circular shape, such as being square or rectangular.

As noted above, the swab 20 may be provided with sealing means such as ridges or fins 27, 28. Alternatively, or in addition, sealing means such as one or more O-rings may be provided along the inlet region 34 and/or fluid chamber 36 (especially along the inlet region 34), to provide further sealing between the shaft 21 and the internal wall of the inlet region 34, or possibly between the shaft 25 and the internal wall of the fluid chamber 36.

Further, in the illustrated embodiment, the inlet region 34 (with stop 33) and the outlet conduit 38 are integrally moulded within the body part 30, sharing a common outer wall 31, whereas, as described above, the fluid chamber 36 is formed as an initially-separate pre-filled component that is inserted into the body part 30 during manufacture, in the manner of a cartridge. As shown for example in FIG. 11, suitable mouldings may be provided within the wall 31 of the body part 30 to secure the fluid chamber 36 in place, e.g. to hold it in a press-fit manner. The walls of the fluid chamber 36 may also incorporate annular fins 36a, 36b, by means of which the fluid chamber 36 may be gripped in a coaxial manner within the wall 31 of the body part 30. Accordingly, as illustrated, the internal diameter of the fluid chamber 36 may be smaller than the internal diameter of the inlet region 34 and the outlet conduit 38, or of the common outer wall 41.

As indicated in FIG. 9, the internal diameter of the fluid chamber 36 may be less than the diameter of the absorbent head 26, to cause the absorbent head 26 to be radially compressed upon entering the fluid chamber 36. Such radial compression of the absorbent head 26 aids the release of the collected fluid sample from the absorbent head 26, and the mixing of the fluid sample with the extraction solution 39 within (and released from) the fluid chamber 36.

The outlet conduit 38 includes, or is in fluid communication with, a proximal end of the lateral flow assay strip 40, such that, in use, extraction solution 39 (containing the collected fluid sample) that enters the outlet conduit 38 is able to reach the proximal end of the assay strip 40, and thence flow along the assay strip 40 to execute a lateral flow test.

The body part 30 further comprises a housing 42 in which at least a substantial part of the lateral flow assay strip 40 is contained. In other words, all of the assay strip 40 may be contained within the housing 42; or almost all of the assay strip 40 may be contained within the housing 42, apart from a proximal portion that is within (or exposed to) the outlet conduit 38. The housing 42 has one or more openings 44 through which one or more indicator regions of the lateral flow assay strip 40 can be viewed.

Structurally, as shown in FIGS. 11 and 12, the housing 42 may be formed as opposing first and second parts 42a, 42b between which the assay strip 40 is mounted. In the illustrated embodiment, the housing 42 is connected to the rest of the body part 30 by means of an end part 41 and a bridging part 42′. The end part 41 also serves as an end wall for the outlet conduit 38.

Design-wise, the arrangement of the housing 42 relative to the rest of the body part 30 is broadly in the manner of a pen clip (e.g. of a fountain pen) relative to the rest of the pen. That is to say, the housing 42 (and the assay strip 40 therein) doubles-back on the rest of the body part 30, making the overall device 10 more compact, and also enabling the overall device 10 to be clipped in a user's pocket (e.g. a jacket or coat pocket) prior to, or after, use.

The dimensions of the device are also broadly similar to a typical pen. For instance, when in the configuration shown in FIG. 1, the overall device may be around 100 mm to 150 mm in length, and may preferably be around 120 mm in length. The external diameter of the body part may be around 10 mm to 15 mm, and may preferably be around 12 mm.

The lateral flow assay strip 40 may have any suitable composition in view of the target substance being detected. For example, it may have a composition as outlined in the “background” section above. The indicator regions of the assay strip 40, visible through the opening(s) 44, may include a first indicator region in which a positive test line is generated if the target substance (e.g. target antigen) is present within the extraction solution, and, usually further along the assay strip, a second indicator region in which a control line is generated to confirm that the extraction solution has flowed along the assay strip 40 correctly.

A QR code 46 or other test identification means, and/or instructional or explanatory text or imagery (e.g. in respect of the meaning of the test lines), may also be provided on the housing 42, or elsewhere on the body part 32.

### Method of Use

To perform a lateral flow test, the method performed by the user simply comprises:
removing the sheathed swab 20 from the body part 30;
removing the sheath 50 from the swab 20, e.g. by pulling the tab 54 and tearing off the sheath 50 using the perforations 56, to expose the absorbent head 26;
collecting a fluid sample (e.g. of oral or nasal fluid) using the absorbent head 26; and then
forcibly inserting the absorbent head 26 into the body part 30 (e.g. via the configuration shown in FIG. 9, into the configuration shown in FIGS. 10 and 12), thereby causing the fluid chamber 36 to open, the extraction solution 39 to treat the absorbent head 26, and extraction solution 39 containing the collected fluid sample to enter the outlet conduit 38, reach the proximal end of the lateral flow assay strip 40, and thence flow along the assay strip 40 (by means of capillary flow) to execute a lateral flow test. The test result is then displayed by means of the one or more indicator regions within the opening(s) 44 on the housing 42, after a given period of time.

More particularly, when using the above-described fluid chamber 36 having a first rupturable seal 37a at a proximal end of the fluid chamber 36, and a second rupturable seal 37b at a distal end of the fluid chamber, the first seal 37a is penetrated by the absorbent head 26 upon partial insertion of the swab 20 into the body part 30, and ruptures; and the second seal 37b is penetrated by the absorbent head 26 upon further (i.e. full) insertion of the swab 20 into the body part 30, and ruptures.

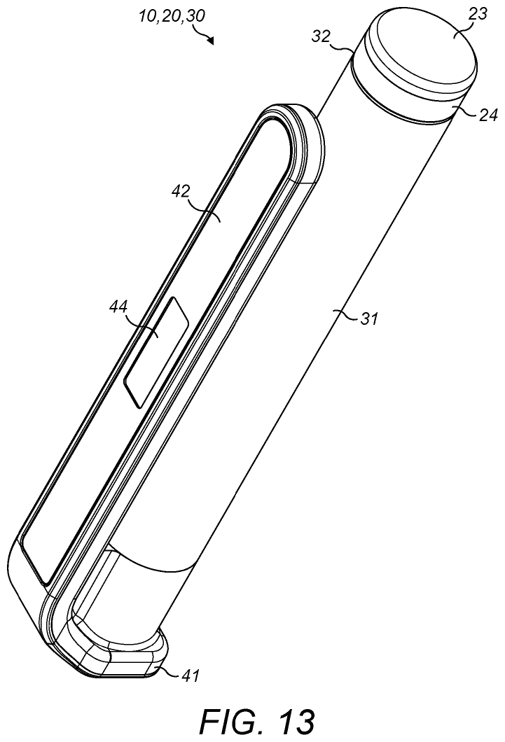

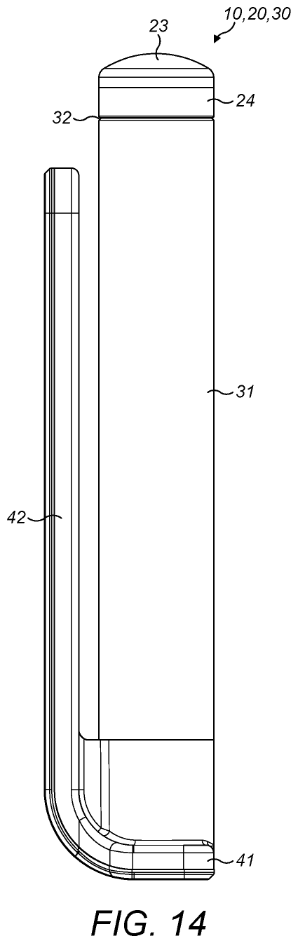

After the lateral flow test has been performed, when the device 10 in the closed configuration as shown in FIGS. 10 and 12 (and FIGS. 13 and 14), the device is 10 is self-sealed by virtue of the above-described sealing means 27, 28 and the plug portion 24b of the handle head 24. This prevents extraction solution 29 from leaking out, and enables the device 10 to be returned to a pocket or the like once the test has been performed. This also prevents the device 10 from drying out for a long while, as the solution 29 is well contained within the device 10.

It will be appreciated that, once the lateral flow test has been performed, the test result remains visible in the indicator region(s) of the assay strip 40 within the opening(s) 44. Accordingly, if the test result is negative, the user will be readily able to show the completed test device 10 to other people, to provide evidence of the user's negative status.

### Summary of Advantages

Compared to a conventional lateral flow device as outlined in the “background” section above, the present lateral flow device 10 provides the following advantages:
the device 10 is provided to the user as a single article (e.g. as shown in FIG. 1), which is compact to store, convenient to carry about one's person, and quick and easy to use;
there is no need for the user to unwrap multiple separate articles prior to use, which helps render the device 10 quick and easy to use (e.g. for people having poor manual dexterity) and significantly reduces packaging waste (as only the sheath 50 needs to be removed prior to use);
there is no need to prepare a clean flat surface on which to place the device 10 for use—instead it can be used substantially anywhere, including when the user is standing up, e.g. away from home;
there is no need for the user to transfer extraction solution to the device 10 from a separate container, thereby removing any risk of spilling the solution, and significantly reducing the risk of contamination;
it is very quick and easy for the user to transfer the collected fluid sample from the swab 20 to the extraction solution 39 (simply by inserting the swab 20 into the body part 30), thereby removing any need to manually squeeze the absorbent head 26 of the swab 20, and again significantly reducing the risk of contamination; and
there is no need for the user to manually transfer a specific quantity (i.e. a specific number of drops) of solution to the lateral flow assay strip, further facilitating the process and again significantly reducing the risk of contamination.

### Modifications and Alternatives

A detailed embodiment and some possible alternatives have been described above. As those skilled in the art will appreciate, a number of modifications and further alternatives can be made to the above embodiment whilst still benefiting from the inventions embodied therein. It will therefore be understood that the invention is not limited to the described embodiment and encompasses modifications apparent to those skilled in the art lying within the scope of the claims appended hereto.

For example, in the illustrated embodiment, the removable sheath 50 comprises a barrier film wrapper. However, in alternative embodiments, the removable sheath may be a substantially rigid cover (e.g. made of any suitable plastics material) that surrounds at least the absorbent head 26 of the swab 20 prior to use. A rigid sheath of this nature may incorporate a collar (similar to collar 52 described above) to removably secure the sheath to the swab 20, and to hold the sheath relative to the inlet region 34 of the body part 30. Similar to the above embodiment, the length of such a sheath would be such as to hold the swab 20 clear of the fluid chamber 36 prior to use.

1. A lateral flow device comprising a body part and a removable swab, wherein the swab comprises a shaft having a proximal end and a distal end, with an absorbent head at the distal end, and the body part comprises:
an inlet region for holding the swab prior to use, and for receiving the swab in use, during the execution of a lateral flow test;
a fluid chamber containing a quantity of extraction solution;
an outlet conduit; and
a lateral flow assay strip;
wherein the fluid chamber is arranged to open upon forcible insertion of the absorbent head into the body part, through the inlet region, in use, to cause the absorbent head to be treated with the extraction solution, and to cause the extraction solution to enter the outlet conduit; and
wherein the outlet conduit includes, or is in fluid communication with, a proximal end of the lateral flow assay strip, such that, in use, extraction solution that enters the outlet conduit is able to reach the proximal end of the lateral flow assay strip, and thence flow along the lateral flow assay strip to execute a lateral flow test.

2. The lateral flow device according to claim 1, wherein the fluid chamber has a first rupturable seal at a proximal end of the fluid chamber, and a second rupturable seal at a distal end of the fluid chamber;
wherein, in use, the first seal of the fluid chamber is arranged to be penetrated by the absorbent head of the swab upon partial insertion of the swab into the body part, and to rupture;
the second seal of the fluid chamber is arranged to be penetrated by the absorbent head of the swab upon further insertion of the swab into the body part, and to rupture; and
the second seal of the fluid chamber is adjacent the outlet conduit, such that, upon rupturing the second seal in use, extraction solution is able to flow from the fluid chamber into the outlet conduit.

3. The lateral flow device according to claim 2, wherein the inlet region, fluid chamber and outlet conduit are arranged colinearly within the body part.

4. The lateral flow device according to claim 2, wherein the inlet region and outlet conduit are integrally formed within the body part, and the fluid chamber is formed as an initially-separate pre-filled component that is inserted into the body part during manufacture.

5. The lateral flow device according to claim 2, wherein the shaft comprises a radially protruding part and the inlet region or the fluid chamber comprises a stop against which the radially protruding part of the swab abuts when the swab is fully inserted into the body part in use.

6. The lateral flow device according to claim 5, wherein the length of the swab from the protruding part to the tip of the absorbent head corresponds to the distance along the body part from the stop to a point partway along the outlet conduit.

7. The lateral flow device according to claim 1, wherein the inlet region is cylindrical;
and/or wherein the fluid chamber is cylindrical;
optionally wherein the internal diameter of the fluid chamber is less than the diameter of the absorbent head.

8. (canceled)

9. (canceled)

10. The lateral flow device according to claim 1, wherein the proximal end of the shaft comprises a handle head by means of which a user can push the swab into the body part in use.

11. The lateral flow device according to claim 10, wherein the handle head has a wider diameter than the inlet region, the handle head having an underside which is shaped to abut the opening of the inlet region when the swab is fully inserted into the body part in use;
optionally wherein the underside of the handle head is further shaped to plug the opening of the inlet region when the swab is fully inserted into the body part in use.

12. (canceled)

13. The lateral flow device according to claim 1, wherein the shaft and/or the inlet region comprise a sealing part for forming a seal between the shaft and the inlet region upon insertion of the absorbent head into the body part in use;
optionally wherein the sealing part comprises one or more fins formed on the shaft.

14. (canceled)

15. The lateral flow device according to claim 1, wherein the body part further comprises a housing in which at least a substantial part of the lateral flow assay strip is contained;
optionally wherein the housing has one or more openings through which one or more indicator regions of the lateral flow assay strip can be viewed;
optionally wherein the arrangement of the housing relative to the rest of the body part is broadly in the manner of a pen clip relative to the rest of a pen.

16. (canceled)

17. (canceled)

18. The lateral flow device according to claim 1, further comprising a removable sheath that surrounds at least the absorbent head of the swab prior to use;
optionally wherein the sheath comprises a film wrapper, or wherein the sheath is substantially rigid.

19. (canceled)

20. (canceled)

21. The lateral flow device according to claim 18, wherein the shaft comprises one or more protrusions, ridges or fins around which a proximal end of the sheath is engaged, prior to use.

22. The lateral flow device according to claim 21, wherein the proximal end of the sheath is formed as a collar around the one or more protrusions, ridges or fins.

23. The lateral flow device according to claim 21, wherein the proximal end of the sheath is provided with a tab and/or perforations by means of which the sheath may be ripped, to enable it to be detached from the swab prior to use.

24. The lateral flow device according to claim 22, wherein the one or more protrusions, ridges or fins, around which the collar is formed, are also configured to form a seal between the shaft and the inlet region upon insertion of the absorbent head into the body part in use.

25. The lateral flow device according to claim 22, wherein the diameter of the collar is greater than the diameter of the inlet region, and the length of the sheath from an underside of the collar to the distal end of the sheath is less than the length of the body part from the opening of the inlet region to the fluid chamber.

26. The lateral flow device according to claim 1, wherein the swab and/or the inlet region comprise a gripping part for releasably gripping the swab within the inlet region prior to use;
optionally wherein the gripping part comprises one or more ridges or fins formed on the shaft.

27. (canceled)

28. A method of performing a lateral flow test using a lateral flow device according to claim 1, the method comprising:
removing the swab from the inlet region of the body part;
collecting a fluid sample using the absorbent head; and
forcibly inserting the absorbent head into the body part, thereby causing the fluid chamber to open, the extraction solution to treat the absorbent head, and extraction solution containing the fluid sample to enter the outlet conduit, reach the proximal end of the lateral flow assay strip, and thence flow along the lateral flow assay strip to execute a lateral flow test.

29. The method according to claim 28, wherein:
the fluid chamber has a first rupturable seal at a proximal end of the fluid chamber, and a second rupturable seal at a distal end of the fluid chamber;
the first seal of the fluid chamber is penetrated by the absorbent head of the swab upon partial insertion of the swab into the body part, and ruptures; and
the second seal of the fluid chamber is penetrated by the absorbent head of the swab upon further insertion of the swab into the body part, and ruptures;
optionally wherein, after removing the swab from the inlet region of the body part and before collecting a fluid sample using the absorbent head, the method further comprises removing a sheath from the swab.

30. (canceled)
